(19) United States
(12) Patent Application Publication
(10) Pub. No.: US 2011/0130567 A1
(43) Pub. Date: Jun. 2, 2011

(54) STEROSELECTIVE SYNTHESIS OF CERTAIN TRIFLUOROMETHYL-SUBSTITUTED ALCOHOLS

(75) Inventors: DANIEL R. FANDRICK, DANBURY, CT (US); JONATHAN T. REEVES, NEW MILFORD, CT (US); JINHUA J. SONG, HOPEWELL JUNCTION, NY (US); ZHULIN TAN, CHESHIRE, CT (US); BO QU, BROOKFIELD, CT (US); NATHAN K. YEE, DANBURY, CT (US); SONIA RODRIGUEZ, NEW MILFORD, CT (US)

(73) Assignee: BOEHRINGER INGELHEIM INTERNATIONAL GMBH, Ingelheim am Rhein (DE)

(21) Appl. No.: 12/788,552

(22) Filed: May 27, 2010

Related U.S. Application Data
(60) Provisional application No. 61/183,606, filed on Jun. 3, 2009.

(51) Int. Cl. C07D 471/04

(52) U.S. Cl. 546/113

(57) ABSTRACT

A process for stereoselective synthesis of a compound of Formula (X)

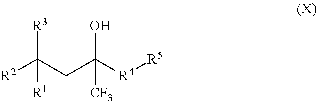

wherein:
R¹ is an aryl group substituted with one to three substituent groups,
   wherein each substituent group of R¹ is independently C₁-C₅ alkyl, aminocarbonyl, alkylaminocarbonyl, dialkylaminocarbonyl, halogen, carboxy, cyano, or trifluoromethyl,
      wherein each substituent group of R¹ is optionally independently substituted with one to three substituents selected from C₁-C₃ alkyl, C₁-C₃ alkoxy, phenyl, and alkoxyphenyl;
R² and R³ are each independently C₁-C₅ alkyl;
R⁴ is C₁-C₅ alkyl optionally independently substituted with one to three substituent groups,
   wherein each substituent group of R⁴ is independently C₁-C₃ alkyl, hydroxy, halogen, amino, or oxo; and
R⁵ is a heteroaryl group substituted with one to three substituent groups,
   wherein each substituent group of R⁵ is independently C₁-C₅ alkyl, aminocarbonyl, alkylaminocarbonyl, dialkylaminocarbonyl, alkylsulfonylamino, aminosulfonyl, C₁-C₅ alkylaminosulfonyl, C₁-C₅ dialkylaminosulfonyl, halogen, hydroxy, carboxy, cyano, trifluoromethyl, trifluoromethoxy, trifluoromethylthio, or C₁-C₅ alkylthio wherein the sulfur atom is optionally oxidized to a sulfoxide or sulfone.

### FIELD OF THE INVENTION

The present invention relates to the stereoselective synthesis of certain trifluoromethyl-substituted alcohols.

### BACKGROUND OF THE INVENTION

Trifluoromethyl-substituted alcohols of formula (I) have been described as ligands that bind to the glucocorticoid receptor. These compounds are potential therapeutics in treating a number of diseases modulated by glucocorticoid receptor function, including inflammatory, autoimmune and allergic disorders. Examples of these compounds are described in U.S. Pat. Nos. 7,268,152; 7,189,758; 7,186,864; 7,074,806; 6,960,581; 6,903,215; and 6,858,627, which are each incorporated herein by reference in their entireties and are hereinafter termed “the Trifluoromethyl-Substituted Alcohol Patent Applications”.

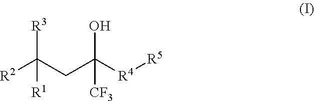

It is well known in the art that enantiomers of a particular compound can have different biological properties including efficacy, toxicity, and pharmacokinetic properties. Thus, it is often desirable to administer one enantiomer of a racemic therapeutic compound.

The synthetic methods disclosed in the patent applications cited above describe the synthesis of racemic products. Separation of enantiomers was accomplished by chiral HPLC and may be accomplished by other conventional ways of separating enantiomers. Chiral HPLC and other enantiomer separation method, however, are generally unsuitable for large-scale preparation of a single enantiomer. Thus, a stereoselective synthesis for preparation of these compounds would be highly desirable.

### SUMMARY OF THE INVENTION

The instant invention is directed to a process for stereoselective synthesis of a compound of Formula (X)

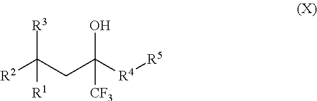

wherein:
R¹ is an aryl group substituted with one to three substituent groups,
   wherein each substituent group of R¹ is independently C₁-C₅ alkyl, aminocarbonyl, alkylaminocarbonyl, dialkylaminocarbonyl, halogen, carboxy, cyano, or trifluoromethyl,
      wherein each substituent group of R¹ is optionally independently substituted with one to three substituents selected from C₁-C₃ alkyl, C₁-C₃ alkoxy, phenyl, and alkoxyphenyl;
R² and R³ are each independently C₁-C₅ alkyl;
R⁴ is C₁-C₅ alkyl optionally independently substituted with one to three substituent groups,
   wherein each substituent group of R⁴ is independently C₁-C₃ alkyl, hydroxy, halogen, amino, or oxo; and
R⁵ is a heteroaryl group substituted with one to three substituent groups,
   wherein each substituent group of R⁵ is independently C₁-C₅ alkyl, aminocarbonyl, alkylaminocarbonyl, dialkylaminocarbonyl, alkylsulfonylamino, aminosulfonyl, C₁-C₅ alkylaminosulfonyl, C₁-C₅ dialkylaminosulfonyl, halogen, hydroxy, carboxy, cyano, trifluoromethyl, trifluoromethoxy, trifluoromethylthio, or C₁-C₅ alkylthio wherein the sulfur atom is optionally oxidized to a sulfoxide or sulfone,
      the process comprising:
(a) reacting the trifluoroacetamide of Formula (A) wherein R′ and R″ are each independently C₁-C₅ alkyl optionally substituted with O or N (e.g., morpholine amide or Weinreb amide) with a vinyl magnesium bromide bearing R² and R³ in a suitable solvent to provide the trifluoromethylenone of Formula (B)

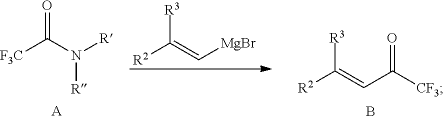

(b) reacting the trifluoromethylenone of Formula (B) with a suitable organocopper reagent generated from an organometallic reagent R⁵R⁴M where M is Li or MgX and a copper salt CuX, where X is Cl, Br, I, or CN in a suitable solvent to form the ketone of Formula (C)

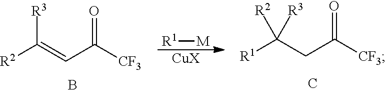

(c) reacting the trifluoromethyl ketone of Formula (C) with an alkyne of Formula (D) in a suitable solvent, in the presence of a suitable base and a metal halide, to obtain a compound of Formula (E)

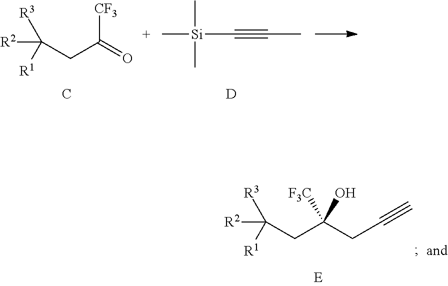

(d) reacting the alkyne of Formula (E) with a protected halopyridylamine of Formula (F), wherein Hal is Br or I, P is an amine protecting group, and R are substituents on R⁵, as set forth above, in a suitable solvent, in the presence of a suitable base and catalyst, to obtain a compound of Formula (X)

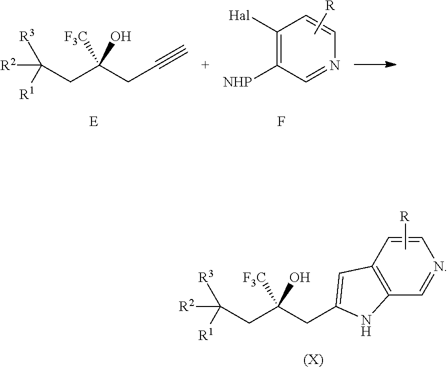

When R¹ is an optionally substituted bromophenyl group, then reaction of a compound of Formula C with an optically active isocyanate of Formula G, in a suitable solvent, in the presence of a suitable base and a suitable organometallic reagent, provides a compound of Formula C′. The compound of Formula C′ may be converted to a compound of Formula (X) by carrying out reactions illustrated in steps (c) and (d) above.

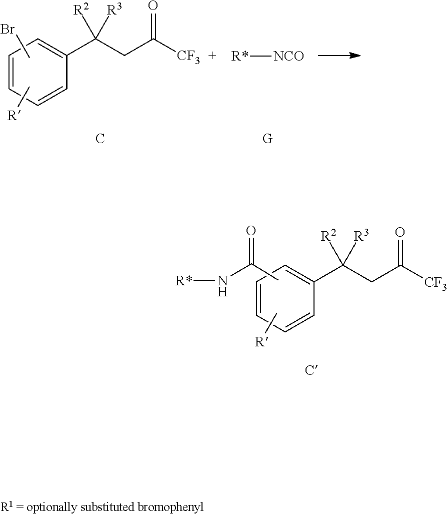

The compound of Formula (X) may be converted to another compound of Formula (X) by reactions known to one skilled in the art.

Another aspect of the invention includes a process for stereoselective synthesis of a compound of Formula (X), wherein:
R¹ is an aryl group substituted with one to three substituent groups,
   wherein each substituent group of R¹ is independently C₁-C₅ alkyl, aminocarbonyl, alkylaminocarbonyl, halogen, carboxy, cyano, or trifluoromethyl,
   wherein each substituent group of R¹ is optionally independently substituted with one to three substituents selected from C₁-C₃ alkyl, phenyl, and alkoxyphenyl;
R² and R³ are each independently C₁-C₃ alkyl;
R⁴ is C₁-C₃ alkyl; and
R⁵ is a heteroaryl group substituted with one to two substituent groups,
   wherein each substituent group of R⁵ is independently aminocarbonyl, alkylaminocarbonyl, dialkylaminocarbonyl, aminosulfonyl, C₁-C₅ alkylaminosulfonyl, C₁-C₅ dialkylaminosulfonyl, or C₁-C₅ alkylthio wherein the sulfur atom is optionally oxidized to a sulfoxide or sulfone,
      the process as set forth above with R¹, R², R³, R⁴, and R⁵ as specified.

In an aspect of the invention, the suitable solvent of step (a) is diethyl ether, dipropyl ether, diisopropyl ether, dibutyl ether, tetrahydrofuran (THF), ethylene glycol dimethyl ether (DME), tert-butyl methyl ether (MTBE), or a mixture thereof, preferably diethyl ether or THF.

In an aspect of the invention, the suitable solvent of step (b) is diethyl ether, dipropyl ether, diisopropyl ether, dibutyl ether, THF, DME, MTBE, toluene, or a mixture thereof, preferably diethyl ether or THF.

In an aspect of the invention, the suitable M of step (b) is Li or MgX, wherein X is Cl, Br, or I.

In an aspect of the invention, the suitable solvent of step (c) is diethyl ether, dipropyl ether, diisopropyl ether, dibutyl ether, THF, DME, MTBE, toluene, or a mixture thereof, preferably diethyl ether or THF. In another aspect of the invention, the suitable solvent for step (c) includes water, preferably at a concentration of 300 to 500 ppm. In yet another aspect of the invention, the suitable solvent for step (c) includes an alcohol, preferably isopropyl alcohol, and preferably the suitable solvent for step (c) includes 4 to 6 mol % of the alcohol compared to substrate R¹, more preferably about 5 mol % of the alcohol compared to substrate R¹.

In another aspect of the invention, the alkyne of step (c) is 1-trimethylsilylpropyne, 1-triethylsilylpropyne, 1-tripropylsilylpropyne, or 1-tert-butyldimethylsilylpropyne. In another aspect of the invention, the suitable base for step (c) is butyllithium or lithium diisopropylamide.

In another aspect of the invention, the process according to claim 1, wherein the metal halide for step (c) is a halide of zinc, magnesium, cerium, barium, or copper, preferably ZnCl₂, ZnBr₂, or ZnI₂.

In still another aspect of the invention, the suitable solvent used in step (d) is methanol, ethanol, isopropanol, THF, MTBE, dimethylformamide, acetonitrile, or dimethylsulfoxide. In another aspect of the invention, the suitable base is triethylamine, tributylamine, pyridine, N-methylpyrroli dine, N-methylpiperidine, 1,8-diazabicyclo[5.4.0]undec-7-ene, or 1,4-diazabicyclo[2.2.2]octane. In another aspect of the invention, the suitable catalyst is palladium acetate, palladium chloride, palladium(allylchloride) dimer, palladium dichlorobis(triphenylphosphine), palladium dichloride bis(acetonitrile), or tetrakis(triphenylphosphine) palladium (0).

In another aspect of the invention, a protected halopyridylamine agent is used in step (d), and the protecting group is tert-butoxycarbonyl, benzyloxycarbonyl, ethyloxycarbonyl, or trifluoroacetyl.

It should be noted that the invention should be understood to include none, some, or all of these various aspects in various combination.

### DETAILED DESCRIPTION OF THE INVENTION

### Definition of Terms and Conventions Used

Terms not specifically defined herein should be given the meanings that would be given to them by one of skill in the art in light of the disclosure and the context. As used in the specification and appended claims, however, unless specified to the contrary, the following terms have the meaning indicated and the following conventions are adhered to.

### A. Chemical Nomenclature, Terms, and Conventions

In the groups, radicals, or moieties defined below, the number of carbon atoms is often specified preceding the group, for example, C₁-C₁₀ alkyl means an alkyl group or radical having 1 to 10 carbon atoms. The term “lower” applied to any carbon-containing group means a group containing from 1 to 8 carbon atoms, as appropriate to the group (i.e., a cyclic group must have at least 3 atoms to constitute a ring). In general, for groups comprising two or more subgroups, the last named group is the radical attachment point, for example, “alkylaryl” means a monovalent radical of the formula Alk-Ar-, while “arylalkyl” means a monovalent radical of the formula Ar-Alk- (where Alk is an alkyl group and Ar is an aryl group). Furthermore, the use of a term designating a monovalent radical where a divalent radical is appropriate shall be construed to designate the respective divalent radical and vice versa. Unless otherwise specified, conventional definitions of terms control and conventional stable atom valences are presumed and achieved in all formulas and groups.

The terms “alkyl” or “alkyl group” mean a branched or straight-chain saturated aliphatic hydrocarbon monovalent radical. This term is exemplified by groups such as methyl, ethyl, n-propyl, 1-methylethyl (isopropyl), n-butyl, n-pentyl, 1,1-dimethylethyl (tert-butyl), and the like. It may be abbreviated “Alk”.

The terms “alkenyl” or “alkenyl group” mean a branched or straight-chain aliphatic hydrocarbon monovalent radical containing at least one carbon-carbon double bond. This term is exemplified by groups such as ethenyl, propenyl, n-butenyl, isobutenyl, 3-methylbut-2-enyl, n-pentenyl, heptenyl, octenyl, decenyl, and the like.

The terms “alkynyl” or “alkynyl group” mean a branched or straight-chain aliphatic hydrocarbon monovalent radical containing at least one carbon-carbon triple bond. This term is exemplified by groups such as ethynyl, propynyl, n-butynyl, 2-butynyl, 3-methylbutynyl, n-pentynyl, heptynyl, octynyl, decynyl, and the like.

The terms “alkylene” or “alkylene group” mean a branched or straight-chain saturated aliphatic hydrocarbon divalent radical having the specified number of carbon atoms. This term is exemplified by groups such as methylene, ethylene, propylene, n-butylene, and the like, and may alternatively and equivalently be denoted herein as -(alkyl)-.

The terms “alkenylene” or “alkenylene group” mean a branched or straight-chain aliphatic hydrocarbon divalent radical having the specified number of carbon atoms and at least one carbon-carbon double bond. This term is exemplified by groups such as ethenylene, propenylene, n-butenylene, and the like, and may alternatively and equivalently be denoted herein as -(alkylenyl)-.

The terms “alkynylene” or “alkynylene group” mean a branched or straight-chain aliphatic hydrocarbon divalent radical containing at least one carbon-carbon triple bond. This term is exemplified by groups such as ethynylene, propynylene, n-butynylene, 2-butynylene, 3-methylbutynylene, n-pentynylene, heptynylene, octynylene, decynylene, and the like, and may alternatively and equivalently be denoted herein as -(alkynyl)-.

The terms “alkoxy” or “alkoxy group” mean a monovalent radical of the formula AlkO—, where Alk is an alkyl group. This term is exemplified by groups such as methoxy, ethoxy, propoxy, isopropoxy, butoxy, sec-butoxy, tert-butoxy, pentoxy, and the like.

The terms “alkoxycarbonyl” or “alkoxycarbonyl group” mean a monovalent radical of the formula AlkO-C(O)—, where Alk is alkyl. Exemplary alkoxycarbonyl groups include methoxycarbonyl, ethoxycarbonyl, tert-butyloxycarbonyl, and the like.

The term “alkoxycarbonylamino” or “alkoxycarbonylamino group” mean a monovalent radical of the formula ROC(O)NH—, where R is lower alkyl.

The terms “alkylcarbonylamino” or “alkylcarbonylamino group” or “alkanoylamino” or “alkanoylamino groups” mean a monovalent radical of the formula AlkC(O)NH—, where Alk is alkyl. Exemplary alkylcarbonylamino groups include acetamido (CH₃C(O)NH—).

The terms “alkylaminocarbonyloxy” or “alkylaminocarbonyloxy group” mean a monovalent radical of the formula AlkNHC(O)O—, where Alk is alkyl.

The terms “amino” or “amino group” mean an —NH₂ group.

The terms “alkylamino” or “alkylamino group” mean a monovalent radical of the formula (Alk)NH—, where Alk is alkyl. Exemplary alkylamino groups include methylamino, ethylamino, propylamino, butylamino, tert-butylamino, and the like.

The terms “dialkylamino” or “dialkylamino group” mean a monovalent radical of the formula (Alk)(Alk)N—, where each Alk is independently alkyl. Exemplary dialkylamino groups include dimethylamino, methylethylamino, diethylamino, dipropylamino, ethylpropylamino, and the like.

The terms “aminocarbonyl”, “alkylaminocarbonyl” or “dialkylaminocarbonyl” mean a monovalent radical of the formula R₂NC(O)—, where the R is independently hydrogen or alkyl.

The terms “substituted amino” or “substituted amino group” mean a monovalent radical of the formula —NR₂, where each R is independently a substituent selected from hydrogen or the specified substituents (but where both R₅ cannot be hydrogen). Exemplary substituents include alkyl, alkanoyl, aryl, arylalkyl, cycloalkyl, heterocyclyl, heteroaryl, heteroarylalkyl, and the like.

The terms “alkoxycarbonylamino” or “alkoxycarbonylamino group” mean a monovalent radical of the formula AlkOC(O)NH—, where Alk is alkyl.

The terms “halogen” or “halogen group” mean a fluoro, chloro, bromo, or iodo group.

The term “halo” means one or more hydrogen atoms of the group are replaced by halogen groups.

The terms “alkylthio” or “alkylthio group” mean a monovalent radical of the formula AlkS—, where Alk is alkyl. Exemplary groups include methylthio, ethylthio, n-propylthio, isopropylthio, n-butylthio, and the like.

The terms “sulfonyl” or “sulfonyl group” mean a divalent radical of the formula —SO₂—.

The terms “aminosulfonyl”, “alkylaminosulfonyl” and “dialkylaminosulfonyl” mean a monovalent radical of the formula R₂N—SO₂—, wherein R is independently hydrogen or alkyl

The terms “aryl” or “aryl group” mean an aromatic carbocyclic monovalent or divalent radical of from 6 to 14 carbon atoms having a single ring (e.g., phenyl or phenylene) or multiple condensed rings (e.g., naphthyl or anthranyl). Unless otherwise specified, the aryl ring may be attached at any suitable carbon atom which results in a stable structure and, if substituted, may be substituted at any suitable carbon atom which results in a stable structure. Exemplary aryl groups include phenyl, naphthyl, anthryl, phenanthryl, indanyl, indenyl, biphenyl, and the like. It may be abbreviated “Ar”.

The term “compounds of the invention” and equivalent expressions are meant to embrace compounds of Formula (I) as herein described, including the tautomers, the prodrugs, the salts, particularly the pharmaceutically acceptable salts, and the solvates and hydrates thereof, where the context so permits. In general and preferably, the compounds of the invention and the formulas designating the compounds of the invention are understood to only include the stable compounds thereof and exclude unstable compounds, even if an unstable compound might be considered to be literally embraced by the compound formula. Similarly, reference to intermediates, whether or not they themselves are claimed, is meant to embrace their salts and solvates, where the context so permits. For the sake of clarity, particular instances when the context so permits are sometimes indicated in the text, but these instances are purely illustrative and it is not intended to exclude other instances when the context so permits.

The terms “optional” or “optionally” mean that the subsequently described event or circumstances may or may not occur, and that the description includes instances where the event or circumstance occurs and instances in which it does not. For example, “optionally substituted aryl” means that the aryl radical may or may not be substituted and that the description includes both substituted aryl radicals and aryl radicals having no substitution.

The terms “stable compound” or “stable structure” mean a compound that is sufficiently robust to survive isolation to a useful degree of purity from a reaction mixture, and formulation into an efficacious therapeutic or diagnostic agent. For example, a compound which would have a “dangling valency” or is a carbanion is not a compound contemplated by the invention.

The term “substituted” means that any one or more hydrogens on an atom of a group or moiety, whether specifically designated or not, is replaced with a selection from the indicated group of substituents, provided that the atom's normal valency is not exceeded and that the substitution results in a stable compound. If a bond to a substituent is shown to cross the bond connecting two atoms in a ring, then such substituent may be bonded to any atom on the ring. When a substituent is listed without indicating the atom via which such substituent is bonded to the rest of the compound, then such substituent may be bonded via any atom in such substituent. For example, when the substituent is piperazinyl, piperidinyl, or tetrazolyl, unless specified otherwise, such piperazinyl, piperidinyl, or tetrazolyl group may be bonded to the rest of the compound of the invention via any atom in such piperazinyl, piperidinyl, or tetrazolyl group. Generally, when any substituent or group occurs more than one time in any constituent or compound, its definition on each occurrence is independent of its definition at every other occurrence. Such combinations of substituents and/or variables, however, are permissible only if such combinations result in stable compounds.

In a specific embodiment, the term “about” or “approximately” means within 20%, preferably within 10%, and more preferably within 5% of a given value or range.

The yield of each of the reactions described herein is expressed as a percentage of the theoretical yield.

### Experimental Examples

The invention provides processes for making compounds of Formula (X). In all schemes, unless specified otherwise, R¹ to R⁵ in the formulas below have the meanings of R¹ to R⁵ in the Summary of the Invention section. Intermediates used in the preparation of compounds of the invention are either commercially available or readily prepared by methods known to those skilled in the art.

The stereoselective synthesis of a compound of Formula (X) is carried out as shown in Scheme I below.

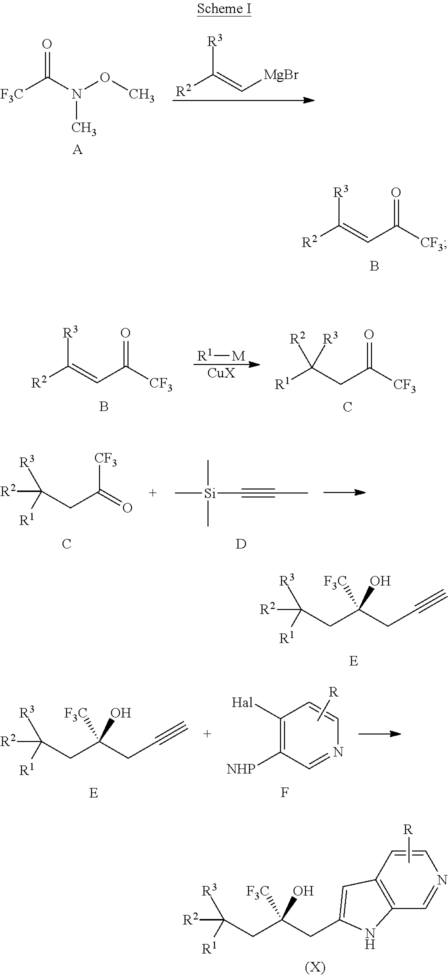

As illustrated in Scheme I, reacting the trifluoroacetamide of Formula (A) with a vinyl magnesium bromide bearing R² and R³ in a suitable solvent provides the trifluoromethylenone of Formula (B). Reacting the trifluoromethylenone of Formula (B) with a suitable organocopper reagent generated from an organometallic reagent R⁵R⁴M where M is Li or MgX and a copper salt CuX, where X is Cl, Br, or I, in a suitable solvent forms the ketone of Formula (C). Reacting the ketone of Formula (C) with an alkyne of Formula (D), in a suitable solvent, in the presence of a suitable base, provides a compound of Formula (E). Reacting the intermediate alkyne of Formula (E) with a protected halopyridylamine of Formula (F), in a suitable solvent, in the presence of a suitable base and catalyst, provides a compound of Formula (X).

Optimum reaction conditions and reaction times may vary depending on the particular reactants used. Unless otherwise specified, solvents, temperatures, pressures, and other reaction conditions may be readily selected by one of ordinary skill in the art. Furthermore, if the substituent groups on R¹ to R⁵ are incompatible under the reaction conditions of the process, protection/deprotection of these groups may be carried out, as required, using reagents and conditions readily selected by one of ordinary skill in the art, see, for example, T. W. Greene and P. G. M. Wuts, Protective Groups in Organic Synthesis, New York: John Wiley & Sons (1999) and references cited therein. For example, a hydroxyl group can be protected as methyl ether and be deprotected at an appropriate stage with reagents, such as boron tribromide in dichloromethane. Specific procedures are provided in the Experimental Examples section. Typically, reaction progress may be monitored by high performance liquid chromatography (HPLC) or thin layer chromatography (TLC), if desired, and intermediates and products may be purified by chromatography on silica gel by recrystallization and/or distillation.

### Synthetic Examples

The following are representative examples that illustrate the process of the invention. HPLC used to determine diastereoselectivity were done on a Supelco SUPELCOSIL™ ABZ+Plus column (4.6 mm×10 cm) eluting with a gradient of 5% acetonitrile/95% water/0.05% TFA to 100% acetonitrile/0.05% TFA over 15 minutes and then held at 100% acetonitrile/0.05% TFA for 5 minutes. References to concentration or evaporation of solutions refer to concentration on a rotary evaporator.

### Example 1

### Synthesis of 2-[3-(5-Methanesulfonyl-1H-pyrrolo[2,3-c]pyridin-2-ylmethyl)-4,4,4-trifluoro-3-hydroxy-1,1-dimethylbutyl]-5-fluoro-N-[(1S)-1-(4-methoxyphenyl)ethyl]benzamide

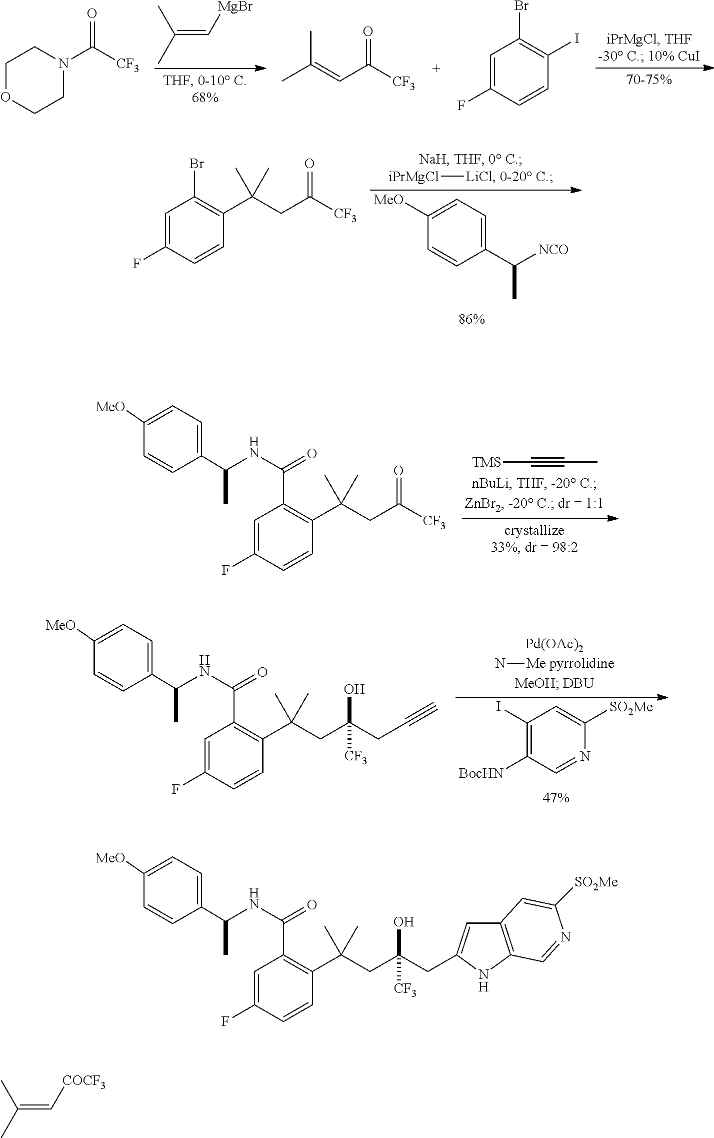

### 1,1,1-trifluoro-4-methyl-3-penten-2-one

2-Methyl-1-propenylmagnesium bromide (0.5M in THF, 2.4 L, 1.2 mol) was cooled to 0° C. N-trifluoroacetylmorpholine (198.1 g, 1.082 mol) was added dropwise over 20 minutes to the Grignard solution at a rate such that the temperature did not exceed 10° C. The reaction mixture was allowed to stir at 15° C.-22° C. for 1 hour. The reaction mixture was cooled to 0° C. and treated dropwise with 300 mL of concentrated hydrochloric acid, keeping the temperature below 30° C. The reaction was further diluted with 900 mL of water and 700 mL of dodecane, and the layers were cut. The organic phase was washed four times with a solution of 1.1 L of water and 300 mL of methanol, then with 1.2 L of water, and finally dried over 100 g of 4 Å molecular sieves for 16 hours. The solution was filtered away from the molecular sieves and distilled at 150 mmHg (bath temperature up to 110° C.) to give 1,1,1-trifluoro-4-methyl-3-penten-2-one (699.3 g, 16 wt. %, thus 111.9 g 1,1,1-trifluoro-4-methyl-3-penten-2-one, 68% yield) as a solution in THF.

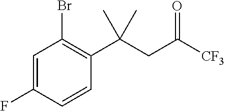

### 4-(2-bromo-4-fluorophenyl)-4-methyl-1,1,1-trifluoropentan-2-one

A solution of 2-bromo-4-fluoro-1-iodobenzene (10.0 g, 33.2 mmol) in 40 mL of THF was cooled to −30° C. and treated dropwise with isopropyl magnesium chloride (17.4 mL, 34.9 mmol, 2.0M/THF) over 15 minutes, keeping the internal temperature between −30° C. to −20° C. After 30 minutes, copper (I) iodide (0.65 g, 3.32 mmol) was added in one portion. The reaction mixture was set aside for 10 minutes at −30° C. 1,1,1-Trifluoro-4-methyl-3-penten-2-one (10.4 g, 33.2 mmol, 48.8 wt. % in THF) was added over 10 minutes, keeping internal temperature between −30° C. to −25° C. The reaction mixture was set aside at −30° C. to −20° C. for 4 hours, and then quenched by addition of 57 mL of 23 wt. % ammonium chloride/water and 22 mL of ethyl acetate. The reaction mixture was stirred at room temperature for 18 hours, and the layers were separated. The organic phase was washed with 22 mL of 23 wt. % ammonium chloride/water and concentrated. Distillation under vacuum (5-15 mmHg) at 85° C.-110° C. gave 4-(2-bromo-4-fluorophenyl)-4-methyl-1,1,1-trifluoropentan-2-one as an orange oil in 70%-75% yield.

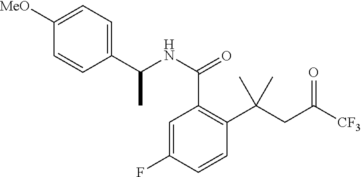

### 5-Fluoro-N-[(1S)-1-(4-methoxyphenyl)ethyl]-2-(4,4,4-trifluoro-1,1-dimethyl-3-oxobutyl)benzamide

A suspension of sodium hydride (6.53 g, 163.1 mmol, 60 wt. %) in 175 mL of THF (containing 300 to 500 ppm water) was cooled to 5° C. A solution of 4-(2-bromo-4-fluorophenyl)-4-methyl-1,1,1-trifluoropentan-2-one (50.0 g, 136.0 mmol, 89.0 wt. %) in THF (25 mL) was added over 5 minutes. The reaction mixture was allowed to stir at 20° C.-25° C. for 1 to 18 hours. The reaction mixture was cooled to 0° C.-5° C. and treated with isopropyl magnesium chloride-lithium chloride (144.8 mL, 153.5 mmol, 1.06 M/THF) over 10 minutes. 1,4-Dioxane (38.5 mL) was added, and the reaction mixture was set aside at 20° C.-25° C. for 2.5 hours, at which time GC analysis showed the Grignard exchange to be >96% complete. The reaction mixture was cooled to 0° C.-5° C., and a solution of (S)-1-(4-methoxyphenyl)ethylisocyanate (26.5 g, 177.2 mmol) in 25 mL of THF was added over 5 minutes. After 15 minutes, the reaction mixture was quenched with 200 mL of aqueous 3N HCl and 150 mL of toluene. The layers were separated, and the organic phase washed with a solution of sodium chloride (7.5 g) in 150 mL of water. The organic phase was concentrated to the minimum volume and 250 mL of heptane and 50 mL of water were charged. The mixture was heated to 70° C., seeded, and allowed to cool to room temperature overnight. The batch was further cooled to 5° C., held at this temperature for 1 hour, filtered, and the solid washed with 200 mL of heptane and dried in a vacuum oven at 50° C. to give 50.5 g of 5-fluoro-N-[(1S)-1-(4-methoxyphenyl)ethyl]-2-(4,4,4-trifluoro-1,1-dimethyl-3-oxobutyl)benzamide, 86% yield.

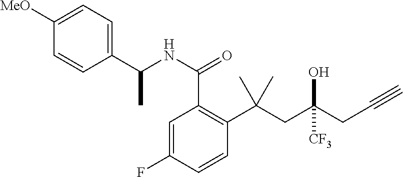

### 5-Fluoro-2-(3-hydroxy-1,1-dimethyl-3-trifluoromethylhex-5-ynyl)-N-[(1S)-1-(4-methoxyphenyl)ethyl]benzamide

A solution 1-trimethylsilylpropyne (6.53 mL, 44.1 mmol) in 50 mL of THF was treated dropwise over 15 minutes with n-BuLi (17.6 mL, 44.1 mmol, 2.5M/hexanes) at −20° C. to −15° C. The reaction mixture was aged at −20° C. for 1 hour and then treated with a solution of zinc bromide in THF (28.4 g, 32.3 mmol, 25.6 wt. %) over 15 minutes, keeping the temperature between −20° C. to −15° C. The reaction mixture was set aside at −20° C. for 1 hour. A solution of 5-fluoro-N-[(1S)-1-(4-methoxyphenyl)ethyl]-2-(4,4,4-trifluoro-1,1-dimethyl-3-oxobutyl)benzamide (12.5 g, 29.38 mmol) in 25 mL of THF was added over 30 minutes, keeping the temperature between −20° C. to −15° C. After 1 hour, the reaction mixture was quenched with 35 mL of methanol and concentrated to 1/3 volume. 35 mL of methanol was added, followed by sodium methoxide (12.7 g, 25 wt. %/MeOH). The reaction mixture was set aside at room temperature for 1 hour. 85% phosphoric acid (3.52 mL) in 25 mL of water was added. An additional 100 mL of water was added followed by 100 mL of ethyl acetate, and the slurry was stirred for 1 hour and filtered. The solid (zinc oxide) was washed with 20 mL of water, then washed with 40 mL of ethyl acetate. The filtrates were combined, and the layers were separated. The organic layer was dried with sodium sulfate and filtered through CELITE® filter aid and brought to a volume of 55 mL by concentration. The solution was seeded, then 70 mL of heptane was added slowly. The mixture was stirred at room temperature for 12 hours, filtered, and the solid washed with heptane and dried to give 4.51 g of 5-fluoro-2-(3-hydroxy-1,1-dimethyl-3-trifluoromethylhex-5-ynyl)-N-[(1S)-1-(4-methoxyphenyl)ethyl]benzamide (33% yield) with a d.r. of 98:2.

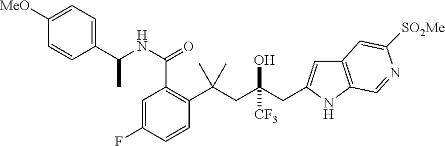

### 2-[3-(5-Methanesulfonyl-1H-pyrrolo[2,3-c]pyridin-2-ylmethyl)-4,4,4-trifluoro-3-hydroxy-1,1-dimethylbutyl]-5-fluoro-N-[(1S)-1-(4-methoxyphenyl)ethyl]benzamide

A solution of 5-fluoro-2-(3-hydroxy-1,1-dimethyl-3-trifluoromethylhex-5-ynyl)-N-[(1S)-1-(4-methoxyphenyl)ethyl]benzamide (110 mg, 0.228 mmol), 5-(N-Boc-amino)-4-iodo-2-methanesulfonylpyridine (80 mg, 0.201 mmol), palladium acetate (5.0 mg, 0.022 mmol), N-methylpyrrolidine (0.10 mL, 0.910 mmol), and methanol (1.0 mL) was stirred at room temperature for 1 hour. Then 1,8-diazabicyclo[5.4.0]undec-7-ene (0.10 mL, 0.658 mmol) was added, and the reaction mixture was stirred at room temperature for 24 hours. The reaction mixture was concentrated and purified by flash column chromatography to give 68 mg of 2-[3-(5-methanesulfonyl-1H-pyrrolo[2,3-c]pyridin-2-ylmethyl)-4,4,4-trifluoro-3-hydroxy-1,1-dimethylbutyl]-5-fluoro-N-[(1S)-1-(4-methoxyphenyl)ethyl]benzamide as a white solid (47% yield).

The following compounds were made according to the above procedure:
2-[3-(5-Methanesulfonyl-1H-pyrrolo[2,3-c]pyridin-2-ylmethyl)-4,4,4-trifluoro-3-hydroxy-1,1-dimethylbutyl]-N-[(1S)-1-(4-methoxyphenyl)ethyl]benzamide;
2-[3-(5-ethanesulfonyl-1H-pyrrolo[2,3-c]pyridin-2-ylmethyl)-4,4,4-trifluoro-3-hydroxy-1,1-dimethylbutyl]-5-fluoro-N-[(1S)-1-(4-methoxyphenyl)ethyl]benzamide; and
2-[3-(5-ethanesulfonyl-1H-pyrrolo[2,3-c]pyridin-2-ylmethyl)-4,4,4-trifluoro-3-hydroxy-1,1-dimethylbutyl]-N-[(1S)-1-(4-methoxyphenyl)ethyl]benzamide.

What is claimed is:

1. A process for stereoselective synthesis of a compound of Formula (X) 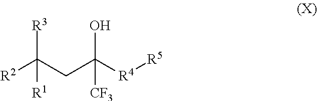
wherein:
R¹ is an aryl group substituted with one to three substituent groups,
wherein each substituent group of R¹ is independently C₁-C₅ alkyl, aminocarbonyl, alkylaminocarbonyl, dialkylaminocarbonyl, halogen, carboxy, cyano, or trifluoromethyl,
wherein each substituent group of R¹ is optionally independently substituted with one to three substituents selected from C₁-C₃ alkyl, C₁-C₃ alkoxy, phenyl, and alkoxyphenyl;
R² and R³ are each independently C₁-C₅ alkyl;
R⁴ is C₁-C₅ alkyl optionally independently substituted with one to three substituent groups,
wherein each substituent group of R⁴ is independently C₁-C₃ alkyl, hydroxy, halogen, amino, or oxo; and
R⁵ is a heteroaryl group substituted with one to three substituent groups,
wherein each substituent group of R⁵ is independently C₁-C₅ alkyl, aminocarbonyl, alkylaminocarbonyl, dialkylaminocarbonyl, alkylsulfonylamino, aminosulfonyl, C₁-C₅ alkylaminosulfonyl, C₁-C₅ dialkylaminosulfonyl, halogen, hydroxy, carboxy, cyano, trifluoromethyl, trifluoromethoxy, trifluoromethylthio, or C₁-C₅ alkylthio wherein the sulfur atom is optionally oxidized to a sulfoxide or sulfone,
the process comprising:
(a) reacting the trifluoroacetamide of Formula (A) wherein R′ and R″ are each independently C₁-C₅ alkyl optionally substituted with O or N (e.g., morpholine amide or Weinreb amide) with a vinyl magnesium bromide bearing R² and R³ in a suitable solvent to provide the trifluoromethylenone of Formula (B) 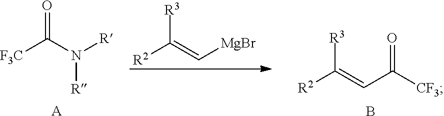
(b) reacting the trifluoromethylenone of Formula (B) with a suitable organocopper reagent generated from an organometallic reagent R⁵R⁴M where M is Li or MgX and a copper salt CuX, where X is Cl, Br, I, or CN in a suitable solvent to form the ketone of Formula (C) 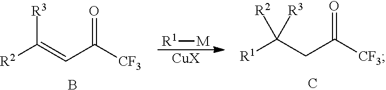
(c) reacting the trifluoromethyl ketone of Formula (C) with an alkyne of Formula (D) in a suitable solvent, in the presence of a suitable base and a metal halide, to obtain a compound of Formula (E) 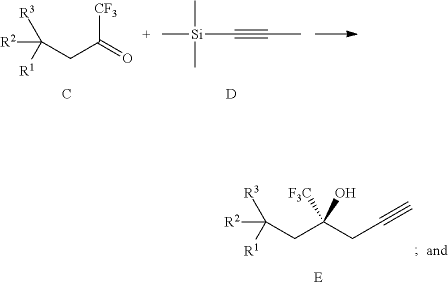
(d) reacting the alkyne of Formula (E) with a protected halopyridylamine of Formula (F), wherein Hal is Br or I, P is an amine protecting group, and R are substituents on R⁵, as set forth above, in a suitable solvent, in the presence of a suitable base and catalyst, to obtain a compound of Formula (X)

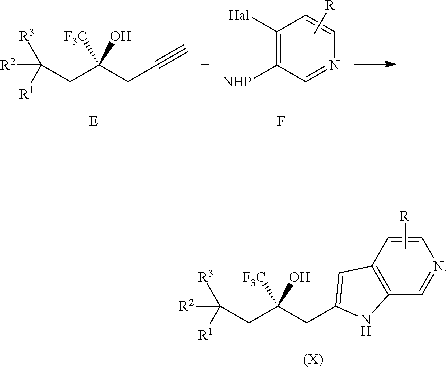

2. The process according to claim 1, wherein:
R¹ is an aryl group substituted with one to three substituent groups,
wherein each substituent group of R¹ is independently C₁-C₅ alkyl, aminocarbonyl, alkylaminocarbonyl, halogen, carboxy, cyano, or trifluoromethyl,
wherein each substituent group of R¹ is optionally independently substituted with one to three substituents selected from C₁-C₃ alkyl, phenyl, and alkoxyphenyl;
R² and R³ are each independently C₁-C₃ alkyl;
R⁴ is C₁-C₃ alkyl; and
R⁵ is a heteroaryl group substituted with one to two substituent groups,
wherein each substituent group of R⁵ is independently aminocarbonyl, alkylaminocarbonyl, dialkylaminocarbonyl, aminosulfonyl, C₁-C₅ alkylaminosulfonyl, C₁-C₅ dialkylaminosulfonyl, or C₁-C₅ alkylthio wherein the sulfur atom is optionally oxidized to a sulfoxide or sulfone.

3. The process according to claim 1, wherein the suitable solvent of step (a) is diethyl ether, dipropyl ether, diisopropyl ether, dibutyl ether, THF, DME, MTBE, or a mixture thereof.

4. The process according to claim 3, wherein the suitable solvent of step (a) is diethyl ether or THF.

5. The process according to claim 1, wherein the suitable solvent of step (b) is diethyl ether, dipropyl ether, diisopropyl ether, dibutyl ether, THF, DME, MTBE, toluene, or a mixture thereof, preferably diethyl ether or THF.

6. The process according to claim 5, wherein the suitable solvent of step (b) is diethyl ether or THF.

7. The process according to claim 1, wherein the suitable M of step (b) is Li or MgX, wherein X is Cl, Br, or I.

8. The process according to claim 1, wherein the suitable solvent of step (c) is diethyl ether, dipropyl ether, diisopropyl ether, dibutyl ether, THF, DME, MTBE, toluene, or a mixture thereof.

9. The process according to claim 8, wherein the suitable solvent of step (c) is diethyl ether or THF.

10. The process according to claim 8, wherein the suitable solvent for step (c) includes water.

11. The process according to claim 8, wherein the suitable solvent for step (c) includes water at a concentration of 300 to 500 ppm.

12. The process according to claim 8, wherein the suitable solvent for step (c) includes an alcohol.

13. The process according to claim 12, wherein the alcohol is isopropyl alcohol.

14. The process according to claim 8, wherein the suitable solvent for step (c) includes 4 to 6 mol % of the alcohol compared to substrate R¹.

15. The process according to claim 8, wherein the suitable solvent for step (c) includes about 5 mol % of the alcohol compared to substrate R¹.

16. The process according to claim 1, wherein the alkyne of step (c) is 1-trimethylsilylpropyne, 1-triethylsilylpropyne, 1-tripropylsilylpropyne, or 1-tert-butyldimethylsilylpropyne.

17. The process according to claim 1, wherein the suitable base for step (c) is butyllithium or lithium diisopropylamide.

18. The process according to claim 1, wherein the metal halide for step (c) is a halide of zinc, magnesium, cerium, barium, or copper.

19. The process according to claim 1, wherein the suitable solvent used in step (d) is methanol, ethanol, isopropanol, THF, MTBE, dimethylformamide, acetonitrile, or dimethylsulfoxide.

20. The process according to claim 1, wherein the suitable base is triethylamine, tributylamine, pyridine, N-methylpyrrolidine, N-methylpiperidine, 1,8-diazabicyclo[5.4.0]undec-7-ene, or 1,4-diazabicyclo[2.2.2]octane.

21. The process according to claim 1, wherein the suitable catalyst is palladium acetate, palladium chloride, palladium(allylchloride) dimer, palladium dichlorobis(triphenylphosphine), palladium dichloride bis(acetonitrile), or tetrakis(triphenylphosphine) palladium (0).

22. The process according to claim 1, wherein a protected halopyridylamine agent is used in step (d), and the protecting group is tert-butoxycarbonyl, benzyloxycarbonyl, ethyloxycarbonyl, or trifluoroacetyl.
